(19) United States
(12) Patent Application Publication
(10) Pub. No.: US 2002/0109013 A1
(43) Pub. Date: Aug. 15, 2002

(54) Aroma Dispenser

(76) Inventor: Jhon Desrosiers, Los Angeles, CA (US)

Correspondence Address: John Desrosiers, # 495, 11693 San Vicente, Los Angeles, CA, 90049, US

(21) Appl. No.: 10/023,048

(22) Filed: Dec. 12, 2001

Related U.S. Application Data
(60) Provisional application No. 60/255,325, filed on Dec. 13, 2000.

(51) Int. Cl. A24F 25/00; A61L 9/04

(52) U.S. Cl. 239/34; 239/36; 239/43; 239/47; 239/53; 239/57

(57) ABSTRACT

An aroma dispenser designed to be worn or carried on the person is described, as is a procedure for using it. The dispenser is unique in that it vents scent to the atmosphere only when activated by the wearer—typically with only one hand—and is constructed to make resealing the vents extremely convenient.

This application claims the benefit under 35 U.S. 119(e) of U.S. provisional application No. 60/255325 filed on Dec. 13, 2000.

### INVENTOR

John Desrosiers

2144 Dellwood Lane

Los Angeles, Calif. 90077

### BACKGROUND OF THE INVENTION

A need exists for a personal, portable and convenient dispenser for aroma from aromatic substances, for example perfume and aromatic oils.

Prior art for portable aroma dispensers currently falls into three broad categories: (1) containers—with or without stoppers or covers—containing aromatic substances, (2) atomizers for aromatic liquids, and (3) aroma-impregnated substances, open to the air, and carried with the person.

(1.a) stoppered or covered containers (for example, a stoppered vial) must be opened using both hands or using awkward one-handed gestures to access the contents, then manually closed. The manual opening and closing, and the two-handed or awkward nature of the operation make it inconvenient. The user may also neglect to close the container after use, leading to evaporation or exhaustion of the contents.

(1.b) uncovered containers continually emit aroma to their surroundings. Such containers are problematic because they can quickly evaporate or exhaust the aroma-bearing substance, because the wearer's sense of smell quickly learns to ignore constant odors, because the wearer may not wish to broadcast a given aroma to their surroundings, and because it is often desirable to carry more than one aroma but not to mix the smells at the same time (for example, a soothing aroma versus a stimulating one). To extend the evaporation period, uncovered aroma containers often contain a significant amount of aroma-bearing medium, adding undesirable size and weight to the package.

(2) atomizers spray aroma-bearing liquid. They are inconvenient for brief, personal aroma dispensing sessions because—depending where aimed—atomizers diffuse aroma over a larger-than-ideal area and the aroma is not concentrated, or atomizers dispense aroma onto the user's person (for example, cologne spray) where it is subject the same problems as (1.b) above. Also, atomizers tend to use up a significant amount of the aroma-bearing medium each time they are activated.

(3) aroma-impregnated substances (for example, aroma-impregnated wooden jewelry) carried open to the air with the person are problematic for the same reasons as (1.b) above. In addition, substances that slowly release aromas improve the lifetime of the aroma, but tend to do so at the expense of the decreased aroma intensity and quality.

| Related U.S. Pat. Nos. | |
|---|---|
| 5,476,194 | Hippely |
| 5,390,510 | Tirio-Cloonan |
| 5,261,570 | Hippely |
| 4,964,570 | Haiduk |
| 4,950,542 | Barker |
| 4,159,631 | Lee |
| 3,270,525 | Sellers |
| 2,751,764 | Hudes |
| 2,708,138 | Gooken |
| 2,079,043 | Samstag |
| 1,899,165 | Rivet |
| D 141,658 | Rubel |
| D 141,655 | Rubel |
| D 140,109 | Pierce |

### BRIEF SUMMARY OF THE INVENTION

This invention discloses an aroma-dispensing system that overcomes many problems associated with the prior art. In summary, it consists of an aroma-bearing substance contained in a relatively small, closed container that can be held in proximity to the user's nose, opened with one or two hands, or with a gesture, held in proximity to the user's nose to dispense aroma, and later closed automatically or with a gesture. As used herein, “aroma-bearing substances” includes, but is not limited to, aromatic liquids, aromatic solids, and appropriate solids impregnated with, containing or holding aromatic liquids or solids.

### BRIEF DESCRIPTION OF THE SEVERAL VIEWS OF THE DRAWING

FIGS. 1A and 1B are sectional views of an example of the first embodiment, in the “closed” and “open” positions respectively. The sectional view is through the center of the apparatus, along the long axis.

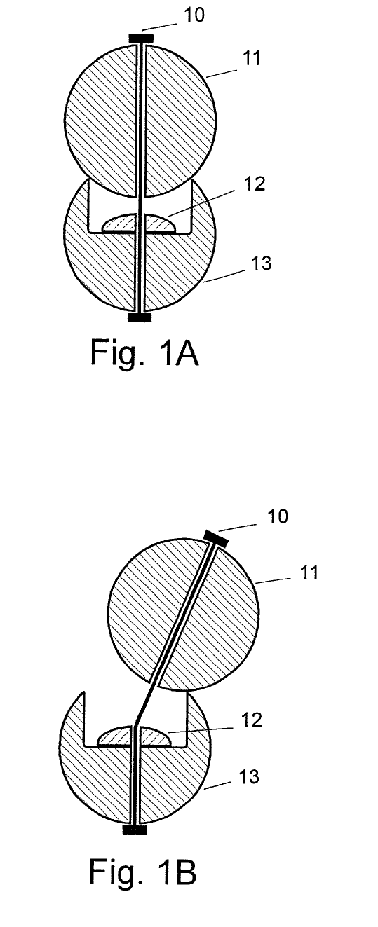

FIG. 2 is a sectional view of an example of the second embodiment, in the “closed” position. The sectional view is through the center of the apparatus, along the long axis.

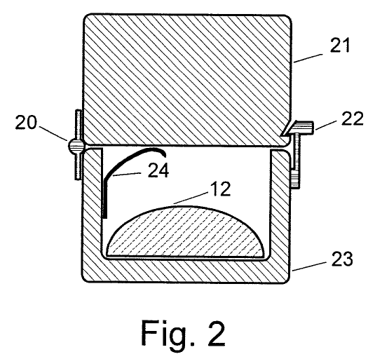

FIGS. 3A and 3B are sectional views of an example of the third embodiment, in the “closed” and “open” positions respectively. The sectional view is through the center of the apparatus, along the long axis.

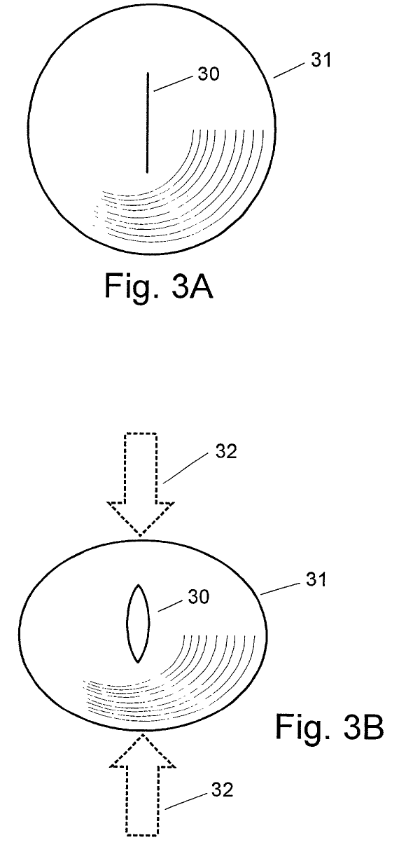

FIG. 4 is a sectional view of an example of the fourth embodiment. The sectional view is through the center of the apparatus, along the long axis.

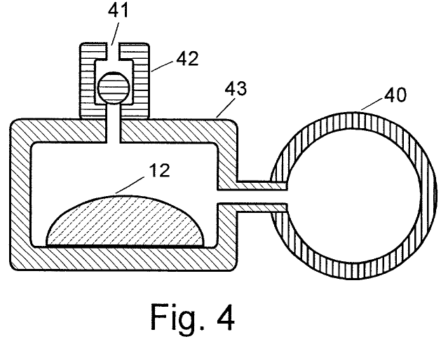

FIGS. 5A and 5B are sectional views of an example of the fifth embodiment, in the “closed” and “open” positions respectively. The sectional view is through the center of the apparatus, along the long axis.

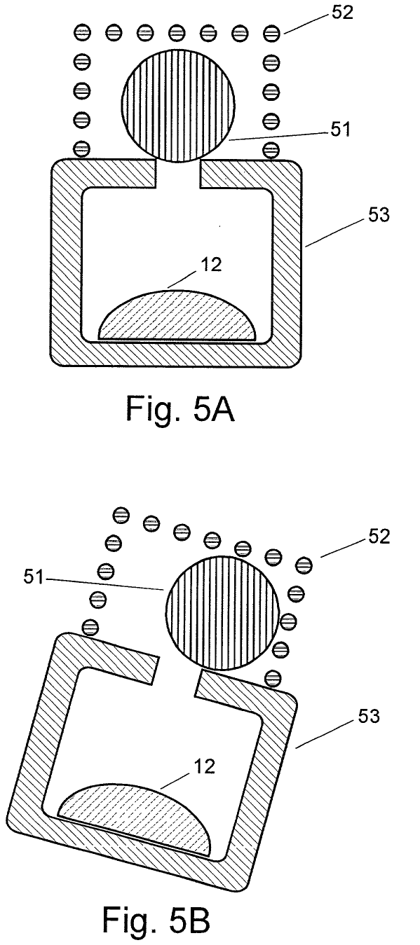

FIGS. 6A and 6B are sectional views of an example of the sixth embodiment, in the “closed” and “open” positions respectively. The sectional view is through the center of the apparatus, along the long axis.

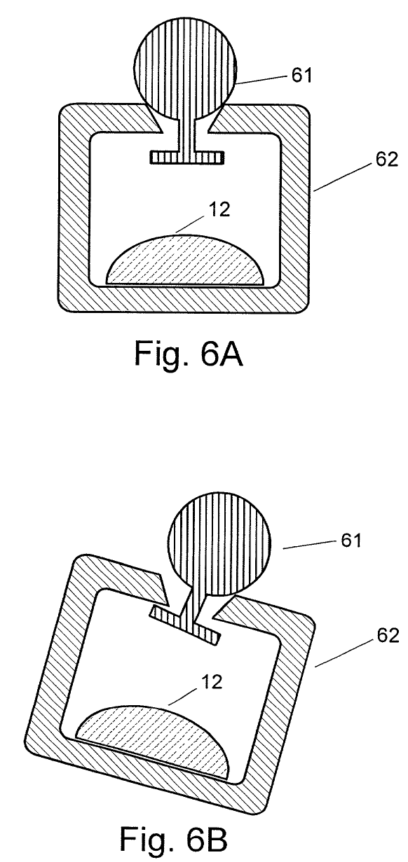

FIGS. 7A and 7B are sectional views of an example of the seventh embodiment, in the “closed” and “open” positions respectively. The sectional view is through the center of the apparatus, along the long axis.

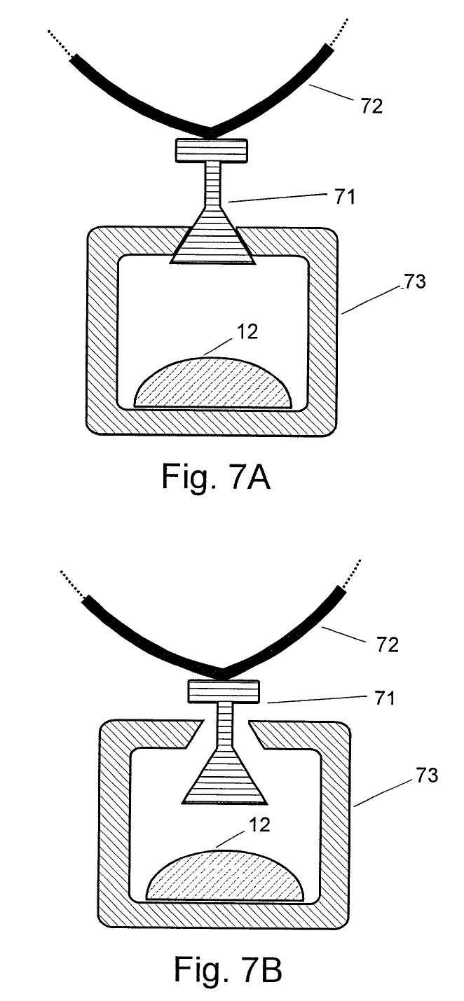

FIGS. 8A and 8B are sectional views of an example of the eighth embodiment, in the “closed” and “open” positions respectively. The sectional view is through the center of the apparatus, along the long axis.

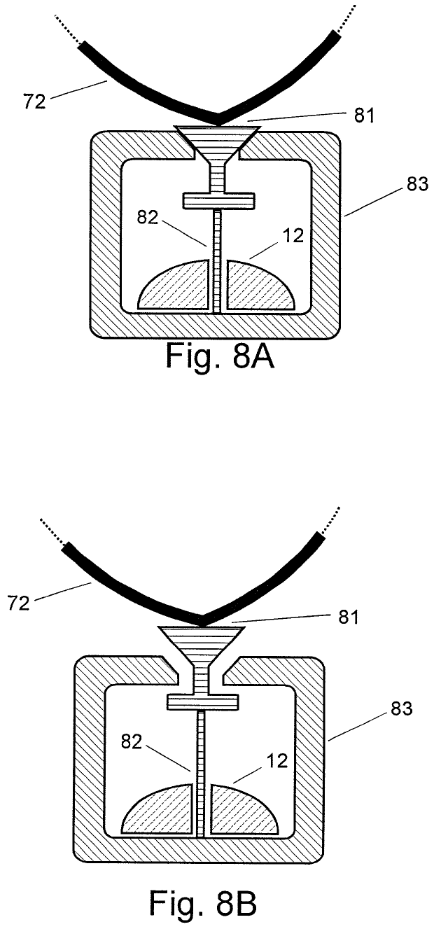

FIGS. 9A and 9B are sectional views of an example of the ninth embodiment, in the “closed” and “open” positions respectively. The sectional view is through the center of the apparatus, along the long axis.

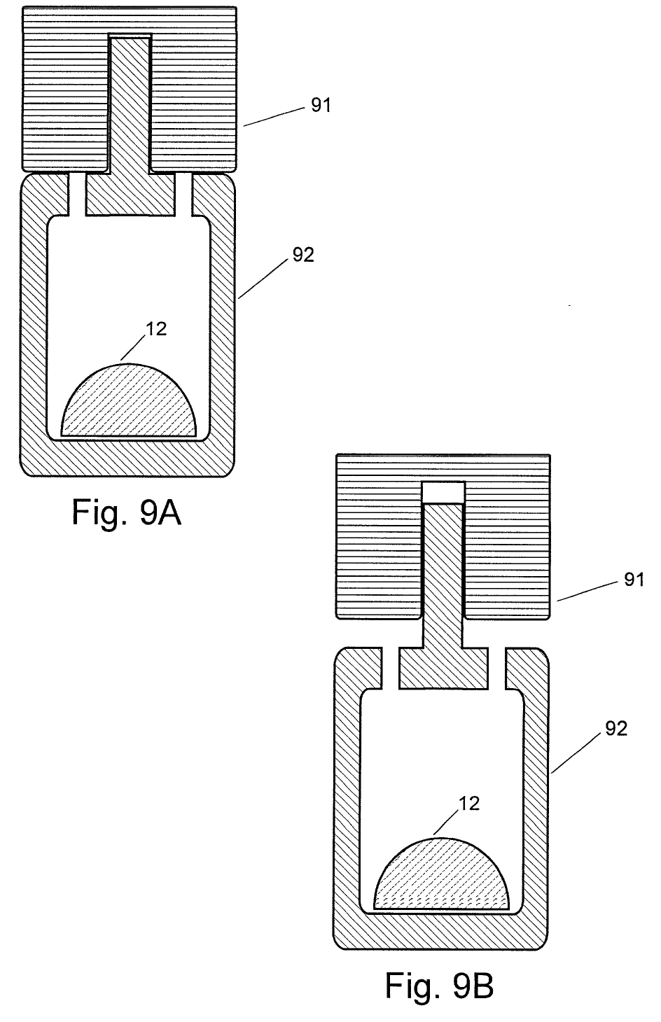

### DETAILED DESCRIPTION OF THE INVENTION

Used herein, the terms “cover” and “vessel” are intended to convey any two matching parts which, together, can enclose a space. The term “elastic” is intended to convey an elastic closure, whether actuated by spring, elastomer, rubber, flexible material, or functionally equivalent means. The terms “open” and “opened” are intended to convey a configuration of vessel and cover such that they allow one or more openings which allow aroma to escape from the enclosed space. The terms “closed” and shut are used interchangably, and are intended to convey a configuration of vessel and cover such that the aroma is substantially confined to the enclosed space. The terms “finger” and “fingers” include the thumb.

The first embodiment consists of a vessel means 13 containing an aroma-bearing substance 12 (for example a piece of sponge wetted with perfume), or having an aroma-bearing inside portion, with a cover means 11 held shut by an elastic closing means 10. The cover and vessel are shaped in such a manner that they can be grasped in one or two hands and held opened, or partially opened, with force from said hand or its fingers. Hand or finger force may be applied directly to the cover and/or vessel, or may be applied via an intermediary mechanical arrangement, for example a lever or can. For example, the dispenser may be designed such that force will separate the cover from tie vessel by pulling one away from the other, by offsetting the cover from the vessel, by hinging the cover and vessel with respect to each other. As a further example, the dispenser could be designed such that force will align openings in the cover and vessel. When the user releases the finger force, the elastic closing mechanism automatically shuts the cover. This aroma dispenser is especially convenient if it can then be safely dropped from the hand and retained close to the person, as is the case if the aroma dispenser is worn on a necklace or bracelet. Note that there could be one or more covers and vessels, or that the cover may also serve as all or part of the vessel and vice versa (for example in the case where the device consists of two half-vessels with an elastic closing mechanism).

The second embodiment consists of a vessel means 23 containing an aroma-bearing substance 12, or having an aroma-bearing inside portion, with a cover means 21 on a hinge means 20 or equivalent mechanism held shut by a latching means 22. The latching means may consist of an identifiable mechanical arrangement, or may simply work by static friction. The cover means can be unlatched by manually unlatching the latching arrangement. The cover means may then be fully or partially opened by an optional spring or elastic or spring mechanism, by flicking the vessel in such a way as to cause the cover means to open, or by opening the cover means with finger force. The dispenser may then be brought in proximity to the nose. The mass of the cover is sufficient so that the container means can be closed by flicking the dispenser and cover assembly in such a way as to swing the cover shut and to latch it. This embodiment is especially well suited to being worn on a bracelet or finger ring because it is convenient to flick the arm, wrist or finger to generate motion to open and close the aroma dispenser. Note that there could be one or more covers, or that the cover may also serve as all or part of the vessel and vice versa.

The third embodiment consists of a vessel means 31, entirely or partially made of flexible material, containing an aroma-bearing substance, or having an aroma-bearing inside portion. Said vessel may consist of a single part or a plurality of parts. There is an opening means 30 in the flexible portion of the device, or at the interface between the flexible and rigid portions of the device, which is normally held closed by the surrounding flexible material,. A simple example of such a device would be a hollow rubber ball containing a small amount of perfume, with a knife slit along a fraction of its circumference. The user can grasp the object in one or two hands and distort the flexible portion of the object by applying appropriate force 32. The force is at least one of: translational, rotational, compressive, or any other force it is possible to generate with one or both hands. The opening in the object will tend to open when distorted, exposing the inside of the container and its aroma-bearing contents. The user may also hasten the delivery of aroma by further compressing the dispenser and expelling the odor-bearing air within. When the user releases the finger force, the container will regain its original shape. It its original shape, the container will once again be closed. Note that there could be one or more openings.

The fourth embodiment consists of a vessel means 43 containing an aroma-bearing substance 12, or having an aroma-bearing inside portion. The container has one or more (preferably small) openings means 41 which vent to outside the container, either directly or via a valve means 42 which opens when there is positive and/or negative gas force within the vessel. The embodiment also includes a pump means 40 to apply negative or positive gas force to the vessel's interior. The user operates the dispenser by holding the vessel's opening in proximity to the nose, and activating the pump means. In the case where the pump means applies positive force to the vessel, aroma-impregnated gas will exit the vessel via the openings. In the case where the pump means applies negative force to the vessel, aroma-impregnated gas from the vessel will exit the pump means. Neither of these arrangements constitutes an atomizer, since aroma-bearing gas—not liquid—is emitted from the dispenser. Note that the vessel and pump means can be one and the same vessel and vice versa.

The fifth embodiment consists of a vessel means 53 containing an aroma-bearing substance 12, or having an aroma-bearing inside portion, with a cover means 51 held shut by gravity, directly or via a mechanical arrangement. The embodiment also includes a constraining means 52 which allows the cover to open and diffuse aroma-bearing gas when the dispenser is tilted from its shut orientation, yet constrains the cover to shut again when the dispenser is returned to its original orientation. Note that there could be one or more covers, or that the cover may also serve as all or part of the vessel and vice versa.

The sixth embodiment consists of a vessel means 62 containing an aroma-bearing substance 12, or having an aroma-bearing inside portion, with a cover means 61 held shut by gravity, directly or via a mechanical arrangement. The embodiment also includes a constraining means which allows the cover to open and diffuse aroma-bearing gas when the cover is tilted, twisted or raised from its shut position, yet constrains the cover to shut again when the cover is allowed to return to its original position. Note that there could be one or more covers, or that the cover may also serve as all or part of the vessel and vice versa.

The seventh embodiment consists of a vessel means 73 containing an aroma-bearing substance 12, or having an aroma-bearing inside portion, with a cover means 71. Said cover is suspended by a suspension means 72 (for example a necklace). The cover is constructed to support the vessel in such a manner that the weight of the vessel causes the cover to seal against the vessel when said vessel's weight is supported by the cover. The cover is also constructed to allow an opening between the cover and the vessel when the vessel's weight is not supported by the cover. At rest, the dispenser is suspended from the cover and is thus closed, for example when the dispenser is suspended from a necklace. To operate the dispenser, the user lifts the vessel (not the cover) and brings it in proximity to the nose. Because the user lifted the vessel and not the cover, the vessel's weight is no longer supported by the cover—this action open the opening between the cover and vessel, allowing aroma to diffuse. Releasing the vessel causes the cover to once again support the vessel, sealing the vessel. Note that there could be one or more covers, or that the cover may also serve as all or part of the vessel and vice versa.

The eighth embodiment consists of a vessel means 83 containing an aroma-bearing substance 12, or having an aroma-bearing inside portion, with a cover means 81. Said cover is suspended by a suspension means 72 (for example a necklace). The vessel is suspended from the cover by an elastic means 82 which causes the vessel to seal with the cover at rest, but which allows the cover and vessel to partially or fully separate from each other when the vessel is pulled away from the cover. The term “elastic” is meant to convey an elastic closure, whether actuated by spring, elastomer, rubber, flexible material, or functionally equivalent means. To operate the dispenser, the user grasps the vessel and pulls the vessel away from the cover—unsealing the vessel—and into proximity of the nose. Releasing the pull on the vessel causes the elastic means to close the cover and seal it to the vessel once again. Note that there could be one or more covers, or that the cover may also serve as all or part of the vessel and vice versa.

The ninth embodiment consists of a vessel means 92 containing an aroma-bearing substance 12, or having an aroma-bearing inside portion, with a cover means 91. Said vessel and cover assembly is constructed such that the size and shape suitable for holding in one hand, and the cover can be conveniently opened and closed with the same hand that is holding the vessel using one gesture. Finger or hand force from the hand holding the assembly is used is used to open the container, and to close it. The closing mechanism is not automatic, but is nonetheless convenient because operation is one-handed and requires only one gesture.

Applications include but are not limited to:

Bracelets

Necklaces, and other neck pendants

Rings

Jewelry charms

Miscellaneous jewelry

Key rings

Pens

Attached to clothing

Watches

Handbags

Hair accessory

I claim:

1. An aroma dispenser worn or carried on the person, carrying an aroma-bearing substance in a vessel means that can be repeatedly vented to the atmosphere and which subsequently substantially reseals automatically.

2. An aroma dispenser per claim 1 which can be operated with only one hand.

3. An aroma dispenser per claim 1 which automatically substantially reseals by elastic means.

4. An aroma dispenser per claim 1 which can be operated with only one hand and which substantially reseals by elastic means.

5. An aroma dispenser per claim 1 which automatically substantially reseals by gravity action.

6. An aroma dispenser per claim 1 which can be operated with only one hand and which substantially reseals by gravity action.

7. An aroma dispenser per claim 1 which can be grasped with one hand, and which can be conveniently vented and substantially sealed by finger or thumb action of the same hand holding the dispenser.

8. An aroma dispenser per claim 1 which can be grasped with one hand, and which can be conveniently vented by finger or thumb action of the same hand holding the dispenser, and which automatically substantially reseals by elastic means.

9. An aroma dispenser per claim 1 which can be grasped with one hand, and which can be conveniently vented by finger or thumb action of the same hand holding the dispenser, and which automatically substantially reseals by gravity action.

10. An aroma dispenser worn or carried on the person, carrying an aroma-bearing substance in a vessel means that can be repeatedly vented to the atmosphere using one hand, and which can subsequently be substantially resealed using the same single hand.

11. An aroma dispenser per claim 10 which substantially reseals by releasing the aroma dispenser.

12. An aroma dispenser per claim 10 which substantially reseals by releasing finger pressure from the aroma dispenser.

13. An aroma dispenser per claim 10 which substantially reseals by flicking the aroma dispenser.

14. An aroma dispenser per claim 10 which forcibly expels scented air when vented by thumb or finger pressure of one hand.

15. Using an aroma dispenser worn or carried on the person by the following process:
Venting the aroma dispenser to the atmosphere by operating its venting means using one hand,
Allowing scented air to escape from the aroma dispenser,
Substantially resealing the aroma dispenser without using a second hand.

16. Using an aroma dispenser worn or carried on the person per claim 15, where the aroma dispenser automatically substantially reseals by simply releasing it.

17. Using an aroma dispenser worn or carried on the person per claim 15, where the aroma dispenser substantially reseals by flicking it with the same hand used to open the venting means.
